(19) United States
(12) Patent Application Publication
(10) Pub. No.: US 2017/0273824 A1
(43) Pub. Date: Sep. 28, 2017

(54) Flexible Microwavable Heat Pack

(71) Applicant: Philip Charles Gannon, Wellingborough (GB)

(72) Inventor: Philip Charles Gannon, Wellingborough (GB)

(21) Appl. No.: 15/168,265

(22) Filed: May 31, 2016

(30) Foreign Application Priority Data
Mar. 24, 2016 (CN) 201620230610.5

(51) Int. Cl. A61F 7/02

(52) U.S. Cl. CPC A61F 7/02; A61F 2007/0024

(57) ABSTRACT

The utility model relates to a flexible microwavable heat pack. At present, most physiotherapy products for treating cervical diseases are electronic physiotherapy products, all of which have the defects of poor curative effects and being easily influenced by such condition factors as time, site, or the like. The utility model consists of a rectangle pack and a plastic spine. The two ends of the rectangle pack are provided with pull-tabs, the interior of the rectangle pack is filled with composite materials, the plastic spine is arranged at a horizontal middle neutral axis part inside the rectangle pack, the two ends of the surface of the rectangle pack are provided with flanges, and the interior of the flange is filled with composite clay; the two sides of the surface of the rectangle pack are provided with a plurality of convex blocks, and the interior of the convex block is filled with composite clay; and the plastic spine is composed by the connection of a plurality of spine joints imitating the shape of a human spine. When in use, the rectangle pack is folded up and down, and placed in a cervical vertebra part of a human body for heat pack (or cold pack); moreover, the pull-tabs may also be pulled for massage. The flexible microwavable heat pack integrates the functions of health care, heat pack or cold pack and massage, s simple to operate, is convenient to use, has wide application scope, and is beneficial for generalizing.

### CROSS-REFERENCE TO RELATED APPLICATIONS

This application claims priority to and the benefit of the non-provisional patent application number 201620230610.5 titled “Flexible Microwavable Heat Pack”, filed in the State Intellectual Property Office of the People's Republic of China on Mar. 24, 2016. The specification of the above referenced patent application is incorporated herein by reference in its entirety.

### TECHNICAL FIELD

The utility model relates to the field of medical care, and more particularly, to a flexible microwavable heat pack.

### BACKGROUND

At present, most physiotherapy products for treating cervical diseases are electronic physiotherapy products, all of which have the defects of poor curative effects and being easily influenced by such condition factors as time, site, or the like.

### SUMMARY

The utility model aims at providing a flexible microwavable heat pack, which can treat anytime and anywhere without any potential risk and side effect, and integrates the functions of health care, heat pack or cold pack, and massage.

The utility model is composed of a rectangle pack and a plastic spine plastic spine. The two ends of the rectangle pack are provided with pull-tabs, the interior of the rectangle pack is filled with composite materials, the plastic spine is arranged at a horizontal middle neutral axis part inside the rectangle pack, the two ends of the surface of the rectangle pack are provided with flanges, and the interior of the flange is filled with composite clay; the two sides of the surface of the rectangle pack are provided with a plurality of convex blocks, and the interior of the convex block is filled with composite clay; and the plastic spine is composed by the connection of a plurality of spine joints imitating the shape of a human spine, and the connection parts of the spine joints are filled with flavour substances.

The product according to the utility model can be soaked with boiled water or heated by a micro boiler for heat pack; and may also be cooled by ice water or refrigerator for cold pack.

When in use, the rectangle pack is folded up and down, and placed in a cervical vertebra part of a human body for heat pack (or cold pack); moreover, the pull-tabs may also be pulled for massage, so that the objects of health care, heat pack or cold pack can be achieved.

The flexible microwavable heat pack integrates the functions of health care, heat pack or cold pack and massage, s simple to operate, is convenient to use, has wide application scope, and is beneficial for generalizing.

### BRIEF DESCRIPTION OF THE DRAWINGS

FIG. 1 is a block diagram showing the entire structure of the utility model. In the figure, 1 refers to a rectangle pack, 2 refers to a plastic spine, 3 refers to a pull-tab, 4 refers to a flange, and 5 refers to a convex block.

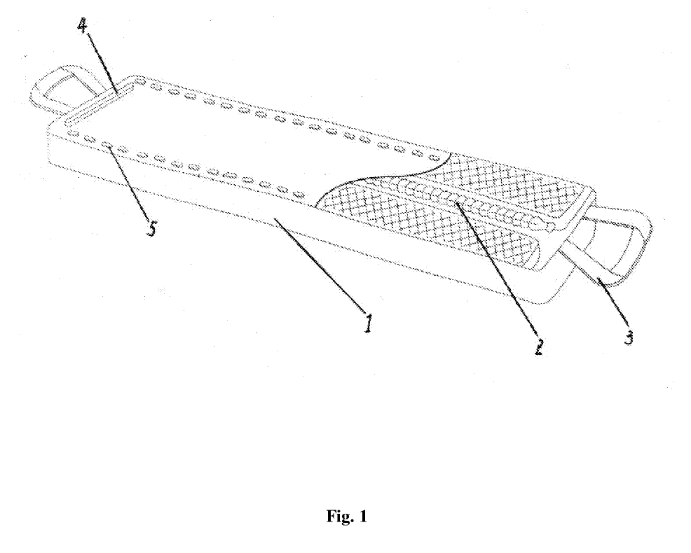

### DETAILED DESCRIPTION

The utility model will be further described in details with reference to the drawings and the best embodiments hereinafter.

The utility model is composed of a rectangle pack (1) and a plastic spine (2). The two ends of the rectangle pack (1) are provided with pull-tabs (3), the interior of the rectangle pack (1) is filled with composite materials, the plastic spine is arranged at a horizontal middle neutral axis part inside the rectangle pack (1), the two ends of the surface of the rectangle pack (1) are provided with flanges (4), and the interior of the flange (4) is filled with composite clay; the two sides of the surface of the rectangle pack (1) are provided with plurality of convex blocks (5), and the interior of the convex block (5) is filled with composite clay; and the plastic spine (2) is composed by the connection of a plurality of spine joints imitating the shape of a human spine, and the connection parts of the spine joints are filled with flavour substances.

The product according to the utility model can be soaked with boiled water or heated by a micro boiler for heat pack; and may also be cooled by ice water or refrigerator for cold pack.

When in use, the rectangle pack (1) is folded up and down, and placed in a cervical vertebra part of a human body for heat pack (or cold pack); moreover, the pull-tabs (3) may also be pulled for massage, so that the objects of health care, heat pack or cold pack can be achieved.

1. A flexible microwavable heat pack, comprising a rectangle pack (1) and a plastic spine (2), wherein the two ends of the rectangle pack (1) are provided with pull-tabs (3), the interior of the rectangle pack (1) is filled with composite materials, the plastic spine (2) is arranged at a horizontal middle neutral axis part inside the rectangle pack (1), the two ends of the surface of the rectangle pack (1) are provided with flanges (4), and the interior of the flange (4) is filled with composite clay; the two sides of the surface of the rectangle pack (1) are provided with a plurality of convex blocks (5), and the interior of the convex block (5) is filled with composite clay; and the plastic spine (2) is composed by the connection of a plurality of spine joints imitating the shape of a human spine, and the connection parts of the spine joints are filled with flavour substances.
